(19) United States
(12) Patent Application Publication
(10) Pub. No.: US 2024/0139193 A1
(43) Pub. Date: May 2, 2024

(54) COMBINATION THERAPY OF KRAS INHIBITOR AND SHP2 INHIBITOR FOR TREATMENT OF CANCERS

(71) Applicant: AMGEN INC., Thousand Oaks, CA (US)

(72) Inventors: Haby HENARY, Moorpark, CA (US); James Russell LIPFORD, Thousand Oaks, CA (US)

(21) Appl. No.: 17/769,251

(22) PCT Filed: Oct. 14, 2020
(86) PCT No.: PCT/US20/55620

§ 371 Date: Apr. 14, 2022

Related U.S. Application Data
(60) Provisional application No. 62/915,564, filed on Oct. 15, 2019.

(51) Int. Cl. A61K 31/519; A61K 31/497; A61P 35/00

(52) U.S. Cl. CPC A61K 31/519; A61K 31/497; A61P 35/00

(57) ABSTRACT

Provided herein are methods of administering a KRAS G12C inhibitor in combination with a SHP2 inhibitor to a cancer subject.

### CROSS-REFERENCE TO RELATED APPLICATIONS

This application claims benefit of U.S. Provisional Patent Application No. 62/915,564, filed Oct. 15, 2019, which is incorporated by reference herein in its entirety.

### FIELD OF INVENTION

Provided herein are methods of administering a KRAS G12C inhibitor in combination with a SHP2 inhibitor to a cancer subject.

### BACKGROUND

KRAS gene mutations are common in pancreatic cancer, lung adenocarcinoma, colorectal cancer, gall bladder cancer, thyroid cancer, and bile duct cancer. KRAS mutations are also observed in about 25% of patients with NSCLC, and some studies have indicated that KRAS mutations are a negative prognostic factor in patients with NSCLC. Recently, V-Ki-ras2 Kirsten rat sarcoma viral oncogene homolog (KRAS) mutations have been found to confer resistance to epidermal growth factor receptor (EGFR) targeted therapies in colorectal cancer; accordingly, the mutational status of KRAS can provide important information prior to the prescription of TKI therapy. Taken together, there is a need for new medical treatments for patients with pancreatic cancer, lung adenocarcinoma, or colorectal cancer, especially those who have been diagnosed to have such cancers characterized by a KRAS mutation and including those who have progressed after chemotherapy. Oncogenic KRAS mutations at residues G12, G13, and Q61 represent the most common RAS mutations found in solid malignancies. Recently it has been demonstrated that KRASG12C can be targeted with covalent small molecule inhibitors which react with the mutant cysteine adjacent to the switch II pocket (SIIP), locking KRAS in its inactive GDP-bound state.

KRAS is the most frequently mutated oncogene in human cancer and encodes a key signaling protein in tumors. The KRASG12C mutant harbors a cysteine that has been exploited to design covalent inhibitors with promising preclinical activity. We optimized a series of inhibitors with novel binding interactions and markedly enhanced potency and selectivity. These efforts led to the discovery of AMG 510 (also referred to as Compound A herein), the first KRASG12C inhibitor in clinical development. Preclinically AMG 510 treatment regressed KRAS p.G12C tumors and significantly improved the anti-tumor efficacy of chemotherapy and targeted agents. In immune-competent mice, AMG 510 treatment resulted in a pro-inflammatory tumor microenvironment and produced durable cures in combination with immune checkpoint inhibition. Cured mice rejected the growth of isogenic KRAS p.G12D tumors, suggesting adaptive immunity against shared antigens. AMG 510 demonstrated preliminary evidence of clinical anti-tumor activity in the first dosing cohort and represents a potentially transformative therapy for patients lacking effective treatments.

The KRAS oncoprotein is a GTPase that is an essential mediator of intracellular signaling pathways involved in tumor cell growth and survival. In normal cells, KRAS functions as a molecular switch, alternating between inactive GDP-bound and active GTP-bound states. Transition between these states is facilitated by guanine nucleotide exchange factors (GEFs) which load GTP and activate KRAS, and GTP hydrolysis, which is catalyzed by GTPase-activating proteins (GAPs) to inactivate KRAS. GTP-binding to KRAS promotes binding of effectors to trigger signal transduction pathways including RAF-MEK-ERK (MAPK). Somatic, activating mutations in KRAS are a hallmark of cancer and prevent the association of GAPs, thereby stabilizing effector-binding and enhancing KRAS signaling. Patients with KRAS mutant tumors have significantly poorer outcomes and worse prognosis. While there are clinically-approved inhibitors of several MAPK pathway proteins (e.g. MEK, BRAF, EGFR) for a subset of tumor types, to date there have been no clinical molecules that are selective for KRAS mutant tumors. Moreover, several MAPK-pathway targeted therapies are contra-indicated for treatment of KRAS mutant tumors due to lack of clinical efficacy. Additionally, non-tumor or non-mutant selective therapies can introduce on-target toxicities due to inhibition of MAPK signaling in normal cells. This might limit the utility for combining such agents with standard-of-care or immunotherapy. Thus, there exists a significant unmet need for the development of tumor-selective therapies that do not introduce liabilities for normal cells.

KRAS p.G12C is present in approximately 13% of lung adenocarcinoma, 3% of colorectal cancer, and 2% of other solid tumors. The mutant cysteine of KRASG12C resides adjacent to a pocket (P2) present in the inactive GDP-bound form of KRAS. The proximity of P2 and a mutant cysteine led to a broad search for covalent inhibitors. The first reported electrophile screen of KRASG12C led to the eventual identification of ARS-1620, which demonstrates in vivo efficacy in preclinical KRAS p.G12C models. While Araxes Pharma's ARS-1620 was a milestone for proof-of-concept, mutant-selective KRAS inhibition, it was positioned as a tool compound for preclinical studies. We identified a series of novel acrylamide-based molecules that utilize a previously unexploited surface groove in KRASG12C to substantially enhance potency and selectivity. Intensive electrophile-screening and structure-based design culminated in the discovery of AMG 510, the first KRASG12C inhibitor to reach clinical testing in humans (See www.clinicaltrials.gov NCT03600883). Here we present compelling clinical activity of AMG 510.

T-cells are activated through a combination of antigen-specific signals from the T-cell receptor (TCR) and antigen-independent signals from the co-stimulatory receptors. In addition, several co-inhibitoly receptors are also expressed on the T-cells which mediate inhibitory signals and balance T-cell activation¹. These co-inhibitory receptors, also known as immune checkpoints, include the programmed cell death 1 (PD-1) and cytotoxic T-lymphocyte-associated protein 4 (CTLA-4), and play important roles in maintaining peripheral tolerance and immune homeostasis². However, this same homeostasis mechanism is utilized by the tumors to escape immune surveillance³. Programmed death-ligand 1 (PD-L1), which is frequently expressed on tumor cells, inhibits naive and effector T-cell responses by binding to PD-1⁴. In recent years, cancer immunotherapy based on blocking PD-1/PD-L1 interaction has shown encouraging results. Immune checkpoint inhibitors like pembrolizumab, nivolumab and atezolizumab have been approved by U.S. Food and Drug Administration (FDA) for the second-line treatment of non-small cell lung cancer (NSCLC). In September 2017, the combination of chemotherapy and Keytruda®/pembrolizumab was approved as the first-line treatment for patients with metastatic non-squamous NSCLC⁵. In addition, other inhibitory receptors such as lymphocyte-activation gene 3 (LAG-3), T cell immunoglobulin (TIM-3), and V-domain Ig suppressor of T cell activation (VISTA) have recently emerged as new potential targets.

A SHP2 inhibitor is an inhibitor of protein tyrosine phosphatase (PTP) non-receptor type 11 (SHP2; src homology region 2 domain phosphatase; PTPN11), with potential antineoplastic activity. Upon administration, a SHP2 inhibitor binds to and inhibits SHP2. This prevents SHP2-mediated signaling, inhibits MAPK signaling and prevents growth of SHP2-expressing tumor cells. SHP2, an oncoprotein overexpressed in a variety of cancer cell types, regulates cell survival, differentiation and proliferation through activation of the RAS-RAF-ERK signaling pathway. SHP2 also regulates programmed cell death 1 (PD-1)-mediated signal transduction and is involved in immune checkpoint modulation.

SHP2 is a non-receptor ubiquitous protein tyrosine phosphatase encoded by the PTPN11 gene in humans, with a relatively conserved structure and function. It contains a protein tyrosine phosphatase catalytic domain (PTP domain), two SH2 domains and a C-terminal tail with two tyrosine phosphorylation sites and a proline-rich motif. SHP2 activity is auto-inhibited by the binding of N-SH2 domain with the PTP domain. Upon stimulation by growth factors or cytokines, the N-SH2 domain binds to specific phospho-tyrosine residues to induce a conformational change, which exposes and catalytically activates the PTP domain, contributing to SHP2 activation and tumorigenesis. As an oncogene, SHP2 regulates cancer cell survival and proliferation primarily by activating the RAS-ERK signaling pathway. Recently, Chen et al. (Y. N. Chen, M. J. LaMarche, H. M. Chan, P. Fekkes, J. Garcia-Fortanet, M. G. Acker, et al. Allosteric inhibition of SHP2 phosphatase inhibits cancers driven by receptor tyrosine kinases Nature, 535 (2016), pp. 148-152), found that cancer cell lines sensitive to SHP2 depletion were also sensitive to EGFR depletion, which validated reports that RTK-driven cancer cells depend on SHP2 for survival. Furthermore, recent studies have shown that SHP2 is required for the growth of mutant KRAS-driven cancers while wild-type KRAS-amplified gastroesophageal cancer can be controlled through combined SHP2 and MEK inhibition.

As a downstream target of several receptors, SHP2 is also involved in signaling in T-cells. It is a downstream molecule in the PD-1 signaling pathway which not only suppresses T-cell activation but also causes T-cell anergy. Previous studies show that SHP2-deficiency in T-cells triggered an anti-tumor immune response against colitis-associated cancer in mice. Therefore, targeting SHP2 may restore or even enhance T-cell functions.

SHP2 inhibitors, such as SHP099, TNO155 (Novartis), RMC-4630 (Revolution Medicines https://clinicaltrials.gov/ct2/show/record/NCT03634982), RMC-4550 (Revolution Medicines) are currently being investigated for cancer treatment and may offer promising results.

The combination of a KRAS G12C inhibitor, such as AMG 510, and a SHP2 inhibitor, such as RMC-4630, may provide enhanced, unexpected results in cancer treatments.

### SUMMARY

Provided herein are methods of treating cancer comprising administering to a subject in need thereof Compound A in a daily dose of 180 mg, 360 mg, 720 mg, or 960 mg in combination with an effective amount of a SHP2 inhibitor, such as RMC-4630. In various cases, the daily dose of Compound A is 180 mg. In various cases, the daily dose of Compound A is 360 mg. In various cases, the daily dose of Compound A is 720 mg. In various cases, the daily dose of Compound A is 960 mg. The dose can be administered orally. The dose can be administered as a single daily dose. The dose can be administered multiple times a day, such as a twice a day, three times a day, or any effective dosing amount. In various cases, the subject is administered Compound A for at least one months, or at least three months, or at least six months.

The subjects administered Compound A in the methods disclosed herein have cancer. The cancer can be a solid tumor. The cancer can be a KRAS G12C mutated cancer. In some cases, the cancer is non-small cell lung cancer. In some cases, the cancer is colorectal cancer. In some cases, the cancer is pancreatic cancer. In various cases, the subject is one who, prior to start of therapy with Compound A, had undergone at least one (e.g., at least two) other systemic cancer therapy.

In various cases, a subject administered Compound A for at least a month does not exhibit any grade 3 or grade 4 adverse events associated with Compound A therapy. In some cases, the subject does not exhibit any grade 3 or grade 4 adverse events associated with Compound A therapy after at least three months of administration of Compound A. In various cases, the subject exhibits an at least stable disease after administration with Compound A. In some cases, the subject exhibits an at least partial response after administration with Compound A.

In various cases, the methods disclosed herein can further comprise administration of a chemotherapeutic. In some cases, the chemotherapeutic comprises an SHP-2 inhibitor. In some cases, the SHP-2 inhibitor is an inhibitor disclosed in WO2018013597A1, Revolution Medicines, Inc., published Jan. 18, 2018). In some cases, the SHP-2 inhibitor is RMC-4630. In some cases, the SHP-2 inhibitor is RMC-4550. In some cases, the SHP-2 inhibitor is SHP099. In some cases, the chemotherapeutic comprises an anti-PD1 antibody. In some cases, the anti-PD1 antibody is Pembrolizumab (Keytruda), Nivolumab, AUNP-12, AMG 404, or Pidilizumab. In some cases, the chemotherapeutic comprises an anti-PDL1 antibody. In some cases, the anti-PDL1 antibody is Atezolizumab, MPDL3280A, Avelumab or Durvalumab. In some cases, the chemotherapeutic comprises a MEK inhibitor. In some cases, the MEK inhibitor is trametinib, pimasertib, PD-325901, MEK162, TAK-733, GDC-0973 or AZD8330. In some cases, the chemotherapeutic comprises a CDK4/6 inhibitor. In some cases, the CDK4/6 inhibitor comprises abemaciclib, or palbociclib. In some cases, the chemotherapeutic comprises a PI3K inhibitor. In some cases, the PI3K inhibitor comprises AMG 511 or buparlisib.

### BRIEF DESCRIPTION OF THE FIGURES

FIG. 1 shows CA 19-9 and CAE biomarker response in patient having metastatic colon adenocarcinoma and administered Compound A at a total daily dose of 360 mg.

FIG. 2 shows non-small cell lung cancer (NSCLC) tumor responses, as measured by radiographic scans every six weeks, for nine NSCLC cancer patients receiving Compound A at various total daily doses as shown.

FIG. 3 shows the response and treatment duration of NSCLC patients administered Compound A at the following total daily doses—top four bars 960 mg; next six bars 720 mg; next bar 360 mg; and bottom three bars 180 mg.

FIG. 4 shows colorectal cancer (CRC) and other solid tumor responses, as measured by radiographic scans every six weeks, for CRC and other solid tumor cancer patients receiving Compound A at various total daily doses as shown.

FIG. 5 shows the response and treatment duration of CRC and Other solid tumor patients administered Compound A at the following total daily doses—top two bars 960 mg; next five bars 720 mg; next eleven bars 360 mg; and bottom three bars 180 mg.

### DETAILED DESCRIPTION

Provided herein are methods of treating cancers by administering Compound A to a subject in need thereof. Compound A has a structure of

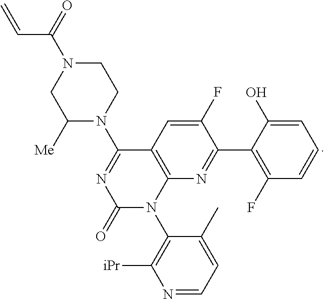 [SMILES: C=CC(=O)N1CCN(c2nc(=O)n(-c3c(C)ccnc3C(C)C)c3nc(-c4c(O)cccc4F)c(F)cc23)C(C)C1]

In some cases, Compound A is referred to as AMG 510. Compound A can be present as a pharmaceutically acceptable isotopically-labeled version, wherein one or more atoms is replaced by atoms having the same atomic number, but an atomic mass or mass number different from the atomic mass or mass number usually found in nature. Examples of isotopes that can be incorporated into Compound A include isotopes of hydrogen, carbon, nitrogen, oxygen, and fluorine, such as ²H, ³H, ¹¹C, ¹³C, ¹⁴C, ¹³N, ¹⁵N, ¹⁵O, ¹⁷O, ¹⁸O, and ¹⁸F, respectively. These radio-labeled compounds could be useful to help determine or measure the effectiveness of Compound A, by characterizing, for example, the site or mode of action, or binding affinity to pharmacologically important site of action. Certain isotopically-labeled versions of Compound A, for example, those incorporating a radioactive isotope, are useful in drug and/or substrate tissue distribution studies. The radioactive isotopes tritium, i.e. ³H, and carbon-14, i.e. ¹⁴ C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art. Isotopically-labeled compounds as disclosed herein can generally be prepared by conventional techniques known to those skilled in the art.

Compound A may exist as a stereoisomer (i.e., isomers that differ only in the spatial arrangement of atoms) including optical isomers and conformational isomers (or conformers). Compound A, when referred to herein unless otherwise indicated, includes all stereoisomers, both as pure individual stereoisomer preparations and enriched preparations of each, and both the racemic mixtures of such stereoisomers as well as the individual diastereomers and enantiomers that may be separated according to methods that are known to those skilled in the art. In some cases, Compound A is provided as 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3]pyrimidin-2(1H)-one:

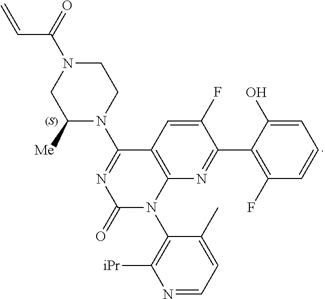

Compound A may exist as an atropisomer, which is a conformational stereoisomer that occur when rotation about a single bond in the molecule is prevented, or greatly slowed, as a result of steric interactions with other parts of the molecule. Compound A, when referred to herein unless otherwise indicated, includes all atropisomers, both as pure individual atropisomer preparations, enriched preparations of each, or a non-specific mixture of each. Where the rotational barrier about the single bond is high enough, and interconversion between conformations is slow enough, separation and isolation of the isomeric species may be permitted. The separation and isolation of the isomeric species is duly designated by the well-known and accepted symbols “M” or “P”. In some cases, Compound A is provided as 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one and the M-atropisomer:

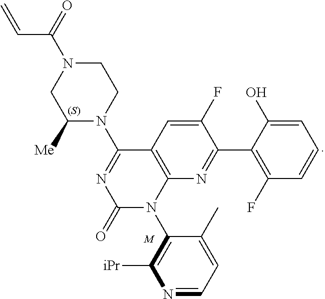

In some cases, Compound A is provided as 4-((R)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one and the M-atropisomer:

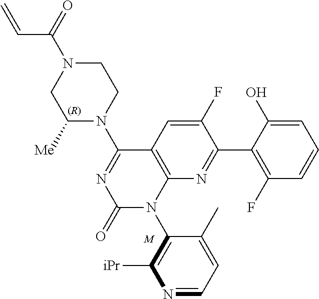

In some cases, Compound A is provided as 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one and the P-atropisomer:

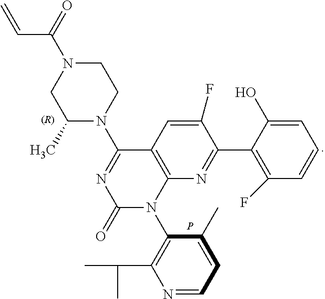

In some cases, Compound A is provided as 4-((R)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one and the P-atropisomer

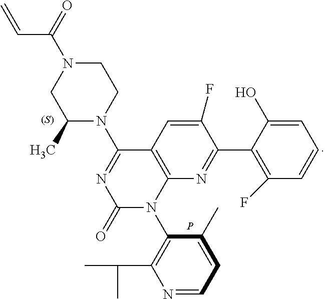

In some cases, Compound A is provided as mixtures of the above isomers.

Compound A can be prepared as reported previously, e.g., generally as disclosed in WO 2018/119183 or specifically as disclosed in WO 2018/217651, incorporated herein by reference in their entireties for all purposes.

Compound A can be provided as a pharmaceutically acceptable salt thereof. Contemplated examples of pharmaceutically acceptable salts include base addition salt and acid addition salts. Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine. Pharmaceutically acceptable acid addition salts include inorganic or organic acid salts. Examples of suitable acid salts include the hydrochlorides, formates, acetates, citrates, salicylates, nitrates, phosphates. Other suitable pharmaceutically acceptable salts are well known to those skilled in the art and include, for example, formic, acetic, citric, oxalic, tartaric, or mandelic acids, hydrochloric acid, hydrobromic acid, sulfuric acid or phosphoric acid; with organic carboxylic, sulfonic, sulfo or phospho acids or N-substituted sulfamic acids, for example acetic acid, trifluoroacetic acid (TFA), propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane 1,2-disulfonic acid, benzenesulfonic acid, 4-methylbenzenesulfonic acid, naphthalene 2-sulfonic acid, naphthalene 1,5-disulfonic acid, 2- or 3-phosphoglycerate, glucose 6-phosphate, N-cyclohexylsulfamic acid (with the formation of cyclamates), or with other acid organic compounds, such as ascorbic acid.

Compound A can be combined with a pharmaceutically acceptable excipient to provide a pharmaceutical formulation (also referred to, interchangeably, as a composition). The excipient can be a diluent or carrier. Suitable pharmaceutical formulations can be determined by the skilled artisan depending on the route of administration and the desired dosage. See, e.g., Remington's Pharmaceutical Sciences, 1435-712 (18th ed., Mack Publishing Co, Easton, Pennsylvania, 1990). Formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data obtainable through animal or human clinical trials. The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable” includes any and all solvents, dispersion media, coatings, antibacterial and antifungal agents, isotonic and absorption delaying agents and the like. The use of such excipients for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin.

Pharmaceutical compositions containing Compound A can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen.

For oral administration, suitable compositions can be formulated readily by combining Compound A with pharmaceutically acceptable excipients such as carriers well known in the art. Such excipients and carriers enable Compound A to be formulated as tablets, pills, dragees, capsules, liquids, gels, syrups, slurries, suspensions and the like, for oral ingestion by a patient to be treated. Pharmaceutical preparations for oral use can be obtained by adding Compound A with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added. Pharmaceutically acceptable ingredients are well known for the various types of formulation and may be for example binders (e.g., natural or synthetic polymers), lubricants, surfactants, sweetening and flavoring agents, coating materials, preservatives, dyes, thickeners, adjuvants, antimicrobial agents, antioxidants and carriers for the various formulation types.

When a therapeutically effective amount of Compound A is administered orally, the composition typically is in the form of a solid (e.g., tablet, capsule, pill, powder, or troche) or a liquid formulation (e.g., aqueous suspension, solution, elixir, or syrup).

When administered in tablet form, the composition can additionally contain a functional solid and/or solid carrier, such as a gelatin or an adjuvant. The tablet, capsule, and powder can contain about 1 to about 95% Compound A, and preferably from about 15 to about 90% Compound A.

When administered in liquid or suspension form, a functional liquid and/or a liquid carrier such as water, petroleum, or oils of animal or plant origin can be added. The liquid form of the composition can further contain physiological saline solution, sugar alcohol solutions, dextrose or other saccharide solutions, or glycols. When administered in liquid or suspension form, the composition can contain about 0.5 to about 90% by weight Compound A, and preferably about 1 to about 50% Compound A. In one embodiment contemplated, the liquid carrier is non-aqueous or substantially non-aqueous. For administration in liquid form, the composition may be supplied as a rapidly-dissolving solid formulation for dissolution or suspension immediately prior to administration.

When a therapeutically effective amount of Compound A is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to Compound A, an isotonic vehicle. Such compositions may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations can optionally contain a preservative to prevent the growth of microorganisms.

Injectable compositions can include sterile aqueous solutions, suspensions, or dispersions and sterile powders for the extemporaneous preparation of sterile injectable solutions, suspensions, or dispersions. In all embodiments the form must be sterile and must be fluid to the extent that easy syringability exists. It must be stable under the conditions of manufacture and storage and must resist the contaminating action of microorganisms, such as bacteria and fungi, by optional inclusion of a preservative. The carrier can be a solvent or dispersion medium containing, for example, water, ethanol, polyol (e.g., glycerol, propylene glycol, and liquid polyethylene glycol, and the like), suitable mixtures thereof, and vegetable oils. In some embodiments contemplated, the carrier is non-aqueous or substantially non-aqueous. The proper fluidity can be maintained, for example, by the use of a coating, such as lecithin, by the maintenance of the required particle size of the compound in the embodiment of dispersion and by the use of surfactants. The prevention of the action of microorganisms can be brought about by various antibacterial and antifungal agents, for example, parabens, chlorobutanol, phenol, sorbic acid, thimerosal, and the like. In many embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating Compound A in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the embodiment of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Slow release or sustained release formulations may also be prepared in order to achieve a controlled release of Compound A in contact with the body fluids in the GI tract, and to provide a substantially constant and effective level of the active compound in the blood plasma. For example, release can be controlled by one or more of dissolution, diffusion, and ion-exchange. In addition, the slow release approach may enhance absorption via saturable or limiting pathways within the GI tract. For example, the compound may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

For administration by inhalation, Compound A is delivered in the form of an aerosol spray presentation from pressurized packs or a nebulizer, with the use of a suitable propellant. In the embodiment of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Compound A can be formulated for parenteral administration by injection (e.g., by bolus injection or continuous infusion). Formulations for injection can be presented in unit dosage form (e.g., in ampules or in multidose containers), with an added preservative. The compositions can take such forms as suspensions, solutions, or emulsions in oily or aqueous vehicles, and can contain formulatory agents such as suspending, stabilizing, and/or dispersing agents.

Pharmaceutical formulations for parenteral administration include aqueous solutions of Compound A in water-soluble form. Additionally, suspensions can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of Compound A and allow for the preparation of highly concentrated solutions. Alternatively, a present composition can be in powder form for constitution with a suitable vehicle (e.g., sterile pyrogen-free water) before use.

Compound A also can be formulated in rectal compositions, such as suppositories or retention enemas (e.g., containing conventional suppository bases). In addition to the formulations described previously, Compound A can be formulated as a depot preparation. Such long-acting formulations can be administered by implantation (e.g., subcutaneously or intramuscularly) or by intramuscular injection. Thus, for example, Compound A can be formulated with suitable polymeric or hydrophobic materials (for example, as an emulsion in an acceptable oil) or ion exchange resins, or as sparingly soluble derivatives, for example, as a sparingly soluble salt.

In particular, Compound A can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound A also can be injected parenterally, for example, intravenously, intramuscularly, subcutaneously, or intracoronarily. For parenteral administration, the compound is best used in the form of a sterile aqueous solution which can contain other substances, for example, salts, or sugar alcohols, such as mannitol, or glucose, to make the solution isotonic with blood.

For veterinary use, Compound A is administered as a suitably acceptable formulation in accordance with normal veterinary practice. The veterinarian can readily determine the dosing regimen and route of administration that is most appropriate for a particular animal.

In some embodiments, all the necessary components for the treatment of KRAS-related disorder using Compound A either alone or in combination with another agent or intervention traditionally used for the treatment of such disease may be packaged into a kit. Specifically, the present invention provides a kit for use in the therapeutic intervention of the disease comprising a packaged set of medicaments that include Compound A as well as buffers and other components for preparing deliverable forms of said medicaments, and/or devices for delivering such medicaments, and/or any agents that are used in combination therapy with Compound A, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the interne.

A “therapeutically effective amount” means an amount effective to treat or to prevent development of, or to alleviate the existing symptoms of, the subject being treated. Determination of the effective amounts is well within the capability of those skilled in the art, especially in light of the detailed disclosure provided herein. Generally, a “therapeutically effective dose” refers to that amount of Compound A that results in achieving the desired effect. For example, a therapeutically effective amount of Compound A decreases KRAS activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

A “therapeutically effective amount” means an amount effective to treat or to prevent development of, or to alleviate the existing symptoms of, the subject being treated. Determination of the effective amounts is well within the capability of those skilled in the art, especially in light of the detailed disclosure provided herein. Generally, a “therapeutically effective dose” refers to that amount of the compound that results in achieving the desired effect. For example, in one preferred embodiment, a therapeutically effective amount of a compound disclosed herein decreases KRAS activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

While individual needs vary, determination of optimal ranges of effective amounts of the compound is within the skill of the art. For administration to a human in the curative or prophylactic treatment of the conditions and disorders identified herein, for example, typical dosages of the compounds of the present invention can be about 0.05 mg/kg/day to about 50 mg/kg/day, for example at least 0.05 mg/kg, at least 0.08 mg/kg, at least 0.1 mg/kg, at least 0.2 mg/kg, at least 0.3 mg/kg, at least 0.4 mg/kg, or at least 0.5 mg/kg, and preferably 50 mg/kg or less, 40 mg/kg or less, 30 mg/kg or less, 20 mg/kg or less, or 10 mg/kg or less, which can be about 2.5 mg/day (0.5 mg/kg×5kg) to about 5000 mg/day (50mg/kg×100kg), for example. For example, dosages of the compounds can be about 0.1 mg/kg/day to about 50 mg/kg/day, about 0.05 mg/kg/day to about 10 mg/kg/day, about 0.05 mg/kg/day to about 5 mg/kg/day, about 0.05 mg/kg/day to about 3 mg/kg/day, about 0.07 mg/kg/day to about 3 mg/kg/day, about 0.09 mg/kg/day to about 3 mg/kg/day, about 0.05 mg/kg/day to about 0.1 mg/kg/day, about 0.1 mg/kg/day to about 1 mg/kg/day, about 1 mg/kg/day to about 10 mg/kg/day, about 1 mg/kg/day to about 5 mg/kg/day, about 1 mg/kg/day to about 3 mg/kg/day, about 1 mg/day to about 960 mg/day, about 20 mg/day to about 720 mg/day, about 3 mg/day to about 500 mg/day, about 5 mg/day to about 360 mg/day, about 10 mg/day to about 100 mg/day, about 3 mg/day to about 10 mg/day, about 100 mg/day to about 250 mg/day. Such doses may be administered in a single dose or it may be divided into multiple doses.

In specific embodiments, Compound A is administered to a subject in need thereof orally and once a day. In specific embodiments, Compound A is administered to a subject in need thereof orally and twice a day. In some cases, the subject is administered a total daily amount of 180 mg, 360 mg, 720 mg, or 960 mg. In some cases, the total daily amount of Compound A administered is 180 mg. In some cases, the total daily amount of Compound A administered is 360 mg. In some cases, the total daily amount of Compound A administered is 720 mg. In some cases, the total daily amount of Compound A administered is 960 mg. In some cases, Compound A is administered in a divided daily dose, such as two, three, four, five, or six times a day.

### EMBODIMENTS

In a first embodiment of the present invention, the present invention comprises a method of treating cancer, the method comprising: administering to a subject in need thereof Compound A in a daily dose of 180 mg, 360 mg, 720 mg, or 960 mg, wherein Compound A has the following structure

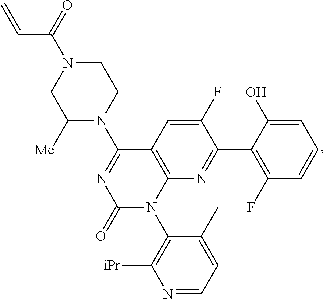 [SMILES: C=CC(=O)N1CCN(c2nc(=O)n(-c3c(C)ccnc3C(C)C)c3nc(-c4c(O)cccc4F)c(F)cc23)C(C)C1]

or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof; in combination with a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof.

In another embodiment, the present invention comprises the method of embodiment 1, wherein Compound A has the structure

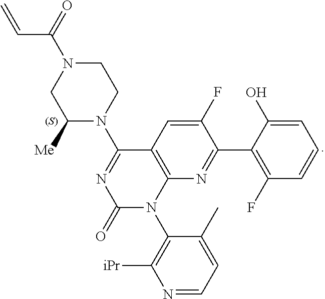

In another embodiment, the present invention comprises the method of embodiment 1, wherein Compound A has the structure

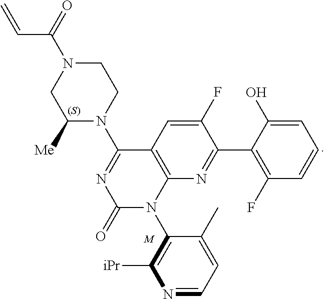

In another embodiment, the present invention comprises the method of any one of embodiments 1, 2 or 3, wherein the SHP2 inhibitor is RMC-4630.

In another embodiment, the present invention comprises the method of any one of embodiments 1-4, wherein the SHP2 inhibitor is orally administered.

In another embodiment, the present invention comprises the method of any one of embodiments 1-5, wherein the cancer is a solid tumor.

In another embodiment, the present invention comprises the method of any one of embodiments 1-6, wherein the cancer is non-small cell lung cancer.

In another embodiment, the present invention comprises the method of any one of embodiments 1-6, wherein the cancer is colorectal cancer.

In another embodiment, the present invention comprises the method of any one of embodiments 1-6, wherein the cancer is pancreatic cancer.

In another embodiment, the present invention comprises the method of any one of embodiments 1 to 9, wherein the cancer is a KRAS G12C mutated cancer.

In another embodiment, the present invention comprises the method of any one of embodiments 1 to 10, wherein the subject, prior to start of Compound A therapy, had undergone at least one other systemic cancer therapy.

In another embodiment, the present invention comprises the method of embodiment 10, wherein the subject had undergone at least two other systemic cancer therapies.

In another embodiment, the present invention comprises the method of any one of embodiments 1 to 10, wherein Compound A is administered orally.

In another embodiment, the present invention comprises the method of any one of embodiments 1 to 13, wherein the Compound A is administered as a single daily dose.

In another embodiment, the present invention comprises the method of any one of embodiments 1 to 13, wherein the Compound A is administered as a twice a day dose.

In another embodiment, the present invention comprises the method of any one of embodiments 1 to 15, wherein the Compound A dose is 180 mg.

In another embodiment, the present invention comprises the method of any one of embodiments 1 to 15, wherein the Compound A dose is 360 mg.

In another embodiment, the present invention comprises the method of any one of embodiments 1 to 15, wherein the Compound A dose is 720 mg.

In another embodiment, the present invention comprises the method of any one of embodiments 1 to 15, wherein the Compound A dose is 960 mg.

In another embodiment, the present invention comprises the method of any one of embodiments 1 to 19, wherein the subject is administered Compound A for at least one month.

In another embodiment, the present invention comprises the method of any one of embodiments 1 to 19, wherein the subject is administered Compound A for at least three months.

In another embodiment, the present invention comprises the method of any one of embodiments 1 to 19, wherein the subject is administered Compound A for at least six months.

In another embodiment, the present invention comprises the method of any one of embodiments 20 to 22, wherein the subject exhibits at least a stable disease (SD).

In another embodiment, the present invention comprises the method of any one of embodiments 20 to 22, wherein the subject exhibits at least a partial response (PR).

In another embodiment, the present invention comprises the method of any one of the embodiments 1-24, wherein the Compound A is administered before the SHP2 inhibitor.

In another embodiment, the present invention comprises the method of any one of the embodiments 1-24, wherein the Compound A is administered concurrently with the SHP2 inhibitor.

In another embodiment, the present invention comprises the method of any one of the embodiments 1-24, wherein the Compound A is administered after the SHP2 inhibitor.

In another embodiment, the present invention comprises a pharmaceutical composition comprising: a Compound A, wherein Compound A has the following structure

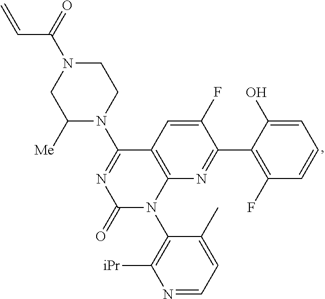 [SMILES: C=CC(=O)N1CCN(c2nc(=O)n(-c3c(C)ccnc3C(C)C)c3nc(-c4c(O)cccc4F)c(F)cc23)C(C)C1]

or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof; and a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof.

In another embodiment, the present invention comprises the composition of embodiment 28, wherein Compound A has the structure

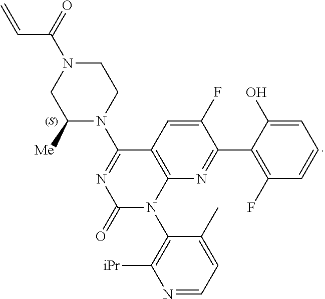

In another embodiment, the present invention comprises the composition of embodiment 28, wherein Compound A has the structure

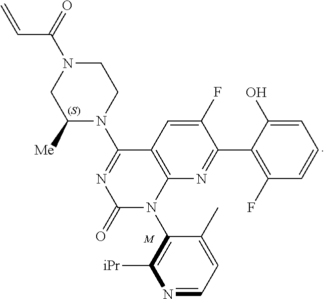

In another embodiment, the present invention comprises a kit for treating cancer, the kit comprising: a Compound A, wherein Compound A has the following structure

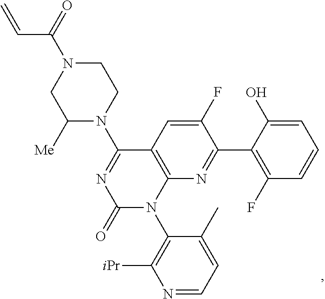 [SMILES: C=CC(=O)N1CCN(c2nc(=O)n(-c3c(C)ccnc3C(C)C)c3nc(-c4c(O)cccc4F)c(F)cc23)C(C)C1]

or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof; and a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof. In another embodiment, the present invention comprises the kit of embodiment 31, wherein Compound A has the structure

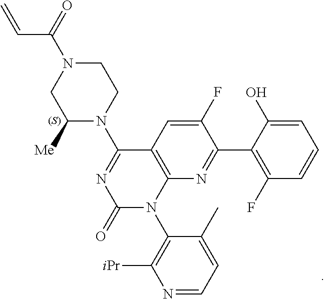

In another embodiment, the present invention comprises the kit of embodiment 31, wherein Compound A has the structure

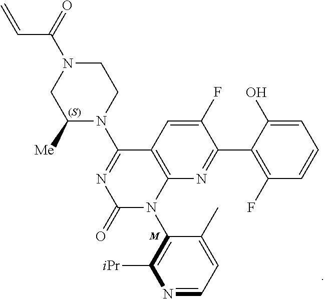

In another embodiment, the present invention comprises the kit of any one of embodiments 31-33, wherein the cancer is a solid tumor.

In another embodiment, the present invention comprises the kit of any one of embodiments 31-33, wherein the cancer is non-small cell lung cancer.

In another embodiment, the present invention comprises the kit of any one of embodiments 31-33, wherein the cancer is colorectal cancer.

In another embodiment, the present invention comprises the kit of any one of embodiments 31-33, wherein the cancer is pancreatic cancer.

In another embodiment, the present invention comprises the kit of any one of embodiments 31-33, wherein the cancer is a KRAS G12C mutated cancer.

### Methods of Using Compound A

In embodiments of the methods disclosed herein, the subject is administered Compound A at a disclosed dose for at least one month, at least six weeks, at least two months, at least three months, at least four months, at least five months, or at least six months.

In some embodiments of the methods disclosed herein, the subject is administered Compound A at a disclosed dose orally at least once daily (QD).

In some embodiments of the methods disclosed herein, the subject is administered Compound A at a disclosed dose orally at least twice daily (BID).

The present disclosure provides a method of inhibiting RAS-mediated cell signaling comprising contacting a cell with an effective amount of Compound A. Inhibition of RAS-mediated signal transduction can be assessed and demonstrated by a wide variety of ways known in the art. Non-limiting examples include a showing of (a) a decrease in GTPase activity of RAS; (b) a decrease in GTP binding affinity or an increase in GDP binding affinity; (c) an increase in k_{off} of GTP or a decrease in k_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf Kits and commercially available assays can be utilized for determining one or more of the above.

The disclosure also provides methods of using Compound A or pharmaceutical compositions of the present disclosure to treat disease conditions, including but not limited to conditions implicated by G12C KRAS, HRAS or NRAS mutation (e.g., cancer).

In some embodiments, a method for treatment of cancer is provided, the method comprising administering an effective amount of Compound A as disclosed herein to a subject in need thereof. In some embodiments, the cancer is mediated by a KRAS, HRAS or NRAS G12C mutation. In various embodiments, the cancer is pancreatic cancer, colorectal cancer or lung cancer (e.g., non-small cell lung cancer). In some embodiments, the cancer is gall bladder cancer, thyroid cancer, and bile duct cancer.

In some embodiments the disclosure provides method of treating a disorder in a subject in need thereof, wherein the said method comprises determining if the subject has a KRAS, HRAS or NRAS G12C mutation and if the subject is determined to have the KRAS, HRAS or NRAS G12C mutation, then administering to the subject a therapeutically effective dose of Compound A or a pharmaceutically acceptable salt thereof.

The disclosed compounds inhibit anchorage-independent cell growth and therefore have the potential to inhibit tumor metastasis. Accordingly, another embodiment the disclosure provides a method for inhibiting tumor metastasis, the method comprising administering an effective amount Compound A.

KRAS, HRAS or NRAS G12C mutations have also been identified in hematological malignancies (e.g., cancers that affect blood, bone marrow and/or lymph nodes). Accordingly, certain embodiments are directed to administration of Compound A (e.g., in the form of a pharmaceutical composition) to a patient in need of treatment of a hematological malignancy. Such malignancies include, but are not limited to, leukemias and lymphomas. For example, Compound A can be used for treatment of diseases such as Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL) and/or other leukemias. In other embodiments, Compound A is useful for treatment of lymphomas such as all subtypes of Hodgkins lymphoma or non-Hodgkins lymphoma. In various embodiments, Compound A is useful for treatment of plasma cell malignancies such as multiple myeloma, mantle cell lymphoma, and Waldenstrom's macroglubunemia.

Determining whether a tumor or cancer comprises a G12C KRAS, HRAS or NRAS mutation can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequence of wild-type human KRAS, HRAS or NRAS is known in the art (e.g. Accession No. NP203524).

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds, and fluorescence is detected. In some embodiments, the KRAS, HRAS or NRAS G12C mutation is identified using a direct sequencing method of specific regions (e.g., exon 2 and/or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

Methods for determining whether a tumor or cancer comprises a G12C KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The disclosure also relates to a method of treating a hyperproliferative disorder in a mammal that comprises administering to said mammal a therapeutically effective amount of Compound A, or a pharmaceutically acceptable salt thereof. In some embodiments, said method relates to the treatment of a subject who suffers from a cancer such as acute myeloid leukemia, cancer in adolescents, adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e. g., psoriasis), restenosis, or prostate (e. g., benign prostatic hypertrophy (BPH)).

In some embodiments, the methods for treatment are directed to treating lung cancers, the methods comprise administering an effective amount of Compound A (or a pharmaceutical composition comprising the same) to a subject in need thereof. In certain embodiments the lung cancer is a non-small cell lung carcinoma (NSCLC), for example adenocarcinoma, squamous-cell lung carcinoma or large-cell lung carcinoma. In some embodiments, the lung cancer is a small cell lung carcinoma. Other lung cancers treatable with the disclosed compounds include, but are not limited to, glandular tumors, carcinoid tumors and undifferentiated carcinomas.

The disclosure further provides methods of modulating a G12C Mutant KRAS, HRAS or NRAS protein activity by contacting the protein with an effective amount of Compound A. Modulation can be inhibiting or activating protein activity. In some embodiments, the disclosure provides methods of inhibiting protein activity by contacting the G12C Mutant KRAS, HRAS or NRAS protein with an effective amount of Compound A in solution. In some embodiments, the disclosure provides methods of inhibiting the G12C Mutant KRAS, HRAS or NRAS protein activity by contacting a cell, tissue, or organ that expresses the protein of interest. In some embodiments, the disclosure provides methods of inhibiting protein activity in subject including but not limited to rodents and mammal (e.g., human) by administering into the subject an effective amount of Compound A. In some embodiments, the percentage modulation exceeds 25%, 30%, 40%, 50%, 60%, 70%, 80%, or 90%. In some embodiments, the percentage of inhibiting exceeds 25%, 30%, 40%, 50%, 60%, 70%, 80%, or 90%.

In some embodiments, the disclosure provides methods of inhibiting KRAS, HRAS or NRAS G12C activity in a cell by contacting said cell with an amount of Compound A sufficient to inhibit the activity of KRAS, HRAS or NRAS G12C in said cell. In some embodiments, the disclosure provides methods of inhibiting KRAS, HRAS or NRAS G12C activity in a tissue by contacting said tissue with an amount of Compound A sufficient to inhibit the activity of KRAS, HRAS or NRAS G12C in said tissue. In some embodiments, the disclosure provides methods of inhibiting KRAS, HRAS or NRAS G12C activity in an organism by contacting said organism with an amount of Compound A sufficient to inhibit the activity of KRAS, HRAS or NRAS G12C in said organism. In some embodiments, the disclosure provides methods of inhibiting KRAS, HRAS or NRAS G12C activity in an animal by contacting said animal with an amount of Compound A sufficient to inhibit the activity of KRAS, HRAS or NRAS G12C in said animal. In some embodiments, the disclosure provides methods of inhibiting KRAS, HRAS or NRAS G12C activity in a mammal by contacting said mammal with an amount of Compound A sufficient to inhibit the activity of KRAS, HRAS or NRAS G12C in said mammal. In some embodiments, the disclosure provides methods of inhibiting KRAS, HRAS or NRAS G12C activity in a human by contacting said human with an amount of Compound A sufficient to inhibit the activity of KRAS, HRAS or NRAS G12C in said human. The present disclosure provides methods of treating a disease mediated by KRAS, HRAS or NRAS G12C activity in a subject in need of such treatment.

### Subject Selection and Therapeutic Results

In some embodiments, the subject being treated by Compound A in the disclosed methods is one who has undergone at least one or more prior systemic cancer therapies (e.g., Compound A is a second or third line therapy). Prior systemic cancer therapies can be any therapy approved by a regulatory authority (e.g., the FDA or EMA) as treatment given type and stage of cancer. In some cases, the prior systemic cancer therapy is a cancer therapy not yet approved by a regulatory authority but undergoing clinical trials. If a subject has had a prior systemic cancer therapy, in some cases, the subject has not undergone any systemic cancer therapy for at least one month, at least two months, at least three months, at least four months, at least five months, or at least six months prior to starting therapy as disclosed herein with Compound A.

in sonic embodiments, the subject will exhibit pathologically documented, locally-advanced or metastatic malignancy with KRAS p. G12C mutation identified through molecular testing. The mutation will be confirmed by central testing prior to enrollment.

In some embodiments, for NSCLC, subjects may have received platinum-based combination therapy and/or targeted therapies (i.e., if molecular testing has identified mutations in EGFR, ALK, or proto-oncogene tyrosine-protein kinase ROS [ROS1] or expression of programmed death-ligand [PD-L1]), prior to receiving AMG 510 (Compound A).

In some embodiments, the NSCLC in subjects must have progressed after receiving anti-PD1 or anti-PD-L1 immunotherapy (unless contraindicated) and/or platinum-based combination chemotherapy and targeted therapy (if actionable oncogenic driver mutations were identified [i.e., EGFR, ALK, and ROS1]). Subjects must have received no more than 3 prior lines of therapy.

In some embodiments for colorectal cancer (CRC), subjects must have received at least 2 prior systemic regimens in the metastatic setting. For those CRC subjects with tumors that are MSI-H, at least 1 of the prior systemic regimens must be treatment with either nivolumab or pembrolizumab if they were clinically able to receive inhibitors and 1 of these agents is approved for that indication in the region or country.

In some embodiments, the CRC in subjects must have progressed after receiving fluoropyrimidine and oxaliplatin and irinotecan. For those CRC subjects with tumors that are MSI-H, at least 1 of the prior systemic regimens must have included an anti-PD1 therapy if they were clinically able to receive inhibitors and 1 of these agents is approved for that indication in the region or country.

In some embodiments, for advanced solid tumor types other than NSCLC or CRC, subjects must have received at least one prior systemic therapy of be intolerant or ineligible for available therapies known to provide clinical benefit.

In some embodiments, dosages of Compound A may optionally be administered to a subject with food, such as consuming a standardized high-fat, high calorie meal, or in a fasting state (no food or liquids, except for water for ≥10 hours).

A subject undergoing a therapy is monitored for adverse events (AE) during the course of the therapy. A treatment related AE is an AE that is related to the treatment drug. A treatment emergent AE is one that a subject develops undergoing the treatment that was not present prior to start of therapy. In some cases, the treatment emergent AE is not or suspected not to be related to the treatment itself. AEs are characterized as one of five grades—grade 1 is a mile AE; grade 2 is a moderate AE; grade 3 is a severe AE; grade 4 is a life-threatening or disabling AE; and grade 5 is death related to AE. In some cases, the subject does not exhibit any grade 3 AE that is treatment related. In some cases, the subject does not exhibit any grade 3 AE. In some cases, the subject does not exhibit any grade 4 AE that is treatment related. In some cases, the subject does not exhibit any grade 4 AE. In various cases, the subject does not exhibit a grade 3 or grade 4 AE that is treatment related after administration of Compound A for at least one month, or at least three months.

In various cases, the subject being treated with Compound A in the methods disclosed herein, does not exhibit any dose limiting toxicities (DLT) at the dose administered. A DLT is any AE meeting the criteria listed below occurring during the first treatment cycle of Compound A (day 1 through day 21) where relationship to the drug cannot be ruled out. The grading of AEs is based on the guidelines provided in the CTCAE version 5.0. AEs for DLT assessment:

Hematological toxicity: Febrile neutropenia; Neutropenic infection; Grade 4 neutropenia; Grade ≥3 thrombocytopenia for >7 days; Grade 3 thrombocytopenia with grade ≥2 bleeding; Grade 4 thrombocytopenia; Grade 4 Anemia Non-hematological toxicity Grade ≥4, vomiting or diarrhea; Grade 3 diarrhea or grade 3 vomiting lasting more than 3 days despite optimal medical support; Grade ≥3 nausea for 3 days or more despite optimal medical support; Any other grade ≥3 AE

In various cases, the subject of the disclosed methods exhibits a response to the therapy. In some cases, the subject exhibits at least a stable disease (SD) due to administration of Compound A. In some cases, the subject exhibits at least a partial response (PR) due to administration of Compound A. The response of a subject is assessed by the criteria as defined by RECIST 1.1, e.g., as discussed in Eisenhauer et al., Eur J Cancer, 45:228-247 (2009). A complete response (CR) is disappearance of all target lesions and any pathological lymph nodes have a reduction in short axis to less than 10 mm. A partial response (PR) is at least a 30% decrease in the sum of diameters of target lesions, taking as reference the baseline sum diameters. A progressive disease is at least a 20% increase in the sum of diameters of target lesions, taking as reference the smallest sum on study (including the baseline sum if that is the smallest on study), and there must be an absolute increase of at least 5 mm in addition to the relative increase of 20%. A stable disease is neither sufficient shrinkage to qualify for PR nor sufficient increase to qualify for PD. A controlled disease state is when a patient may alternate between exhibiting a stable disease and a partial response. The tumor size can be measured by radiographic scan.

### Combination Therapy

The present disclosure also provides methods for combination therapies in which an agent known to modulate other pathways, or other components of the same pathway, or even overlapping sets of target enzymes are used in combination with Compound A, or a pharmaceutically acceptable salt thereof. In one aspect, such therapy includes but is not limited to the combination of Compound A as disclosed herein with a chemotherapeutic agent to provide a synergistic or additive therapeutic effect.

Many chemotherapeutics are presently known in the art and can be used in combination with Compound A. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), Venclexta™ (venetoclax) and Adriamycin™, (docorubicin) as well as a host of chemotherapeutic agents. Non-limiting examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, chlorocyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

Compound A can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Compound A is contemplated for use in co-therapies with a chemotherapeutic that is an anti-neoplastic agent, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-ₙ₃, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Compound A can be used in combination with a chemotherapeutic that is an PD1 inhibitor, PDL1 inhibitor, MEK inhibitor, PI3K inhibitor, or CDK4/6 inhibitor.

The KRAS^{G12C} inhibitors of the present invention can be used in combination with MEK inhibitors. Particular MEK inhibitors that can be used in the combinations of the present invention include PD-325901, trametinib, pimasertib, MEK162 [also known as binirnetinib], TAK-733, GDC-0973 and AZD8330. A particular MEK inhibitor that can be used along with KRAS^{G12C} inhibitor in the combinations of the present invention is trametinib (tardename: Mekinist®, commercially available from Novartis Pharmaceuticals Corp.). Another particular MEK inhibitor is N-(((2R)-2,3-dihydroxypropyl)oxy)-3,4-difluoro-2-((2-fluoro-4-iodophenyl)amino)benzamide, also known as AMG 1009089, 1009089 or PD-325901. Another particular MEK inhibitor that can be used in the combinations of the present invention includes cobimetinib. In some cases, the MEK inhibitor is CI-1040, AZD6244, PD318088, PD98059, PD334581, RDEA119, ARRY-142886, ARRY-438162, or PD-325901.

In another aspect, Compound A can be used in combination with one or more agents that is an inhibitor of a protein in the phosphatidylinositol 3-kinase (PI3K) pathway. Examples of proteins in the PI3K pathway include PI3K, mTOR and PKB (also known as Akt or AKT). The PI3K protein exists in several isoforms including α, β, δ, or γ. It is contemplated that a PI3K inhibitor can be selective for one or more isoform. By selective it is meant that the compounds inhibit one or more isoform more than other isoforms. Selectivity is a concept well known to those is the art and can be measured with well-known in vitro or cell-based activity assays. Preferred selectivity includes greater than 2-fold, preferably 10-fold, or more preferably 100-fold greater selectivity for one or more isoform over the other isoforms. In one aspect, the PI3K inhibitors that can be used in combination with Compound A are PI3K α selective inhibitors. In another aspect the compound is a PI3K δ selective inhibitor. In still another aspect, the compound is a PI3K β selective inhibitor.

Examples of PI3K inhibitors that can be used in combination with Compound A include those disclosed in the following: PCT published application no. WO2010/151791; PCT published application no. WO2010/151737; PCT published application no. WO2010/151735; PCT published application no. WO2010151740; PCT published application no. WO2008/118455; PCT published application no. WO2008/118454; PCT published application no. WO2008/118468; U.S. published application no. US20100331293; U.S. published application no. US20100331306; U.S. published application no. US20090023761; U.S. published application no. US20090030002; U.S. published application no. US20090137581;U.S. published application no. US2009/0054405; U.S. published application no. U.S. 2009/0163489; U.S. published application no. US 2010/0273764; U.S. published application no. U.S. 2011/0092504; or PCT published application no. WO2010/108074.

In particular, PI3K inhibitors include, but are not limited to, wortmannin, 17-hydroxywortmannin analogs described in WO 06/044453, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in PCT Publication No. WO 2008/070740), LY294002 (2-(4-Morpholinyl)-8-phenyl-4H-1-benzopyran-4-one available from Axon Medchem), PI 103 hydrochloride (3-[4-(4-morpholinylpyrido-[3′,2′:4,5]furo[3,2-d]pyrimidin-2-yl]phenol hydrochloride available from Axon Medchem), PIK 75 (N′-[(1E)-(6-bromoimidazo[1,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydro-imidazo[1,2-c]quinazolin-5-yl)-nicotinamide available from Axon Medchem), GDC-0941 bismesylate (2-(1H-Indazol-4-yl)-6-(4-methanesulfonyl-piperazin-1-ylmethyl)-4-morpholin-4-yl-thieno[3,2-d]pyrimidine bismesylate available from Axon Medchem), AS-252424 (5-[1-[5-(4-Fluoro-2-hydroxy-phenyl)-furan-2-yl]-meth-(Z)-ylidene]-thiazolidine-2,4-dione available from Axon Medchem), and TGX-221 (7-Methyl-2-(4-morpholinyl)-9-[1-(phenylamino)ethyl]-4H-pyrido-[1,2-a]pyrimidin-4-one available from Axon Medchem), XL-765, and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TG100-115, CAL263, PI-103, GNE-477, CUDC-907, and AEZS-136.

Preferred PI3K inhibitors for use in combination with the compound of the present invention include:

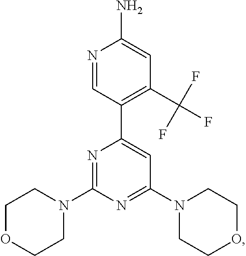 [SMILES: Nc1cc(C(F)(F)F)c(-c2cc(N3CCOCC3)nc(N3CCOCC3)n2)cn1]

also known as buparlisib, an investigational small molecule from Novartis Pharmaceuticals,

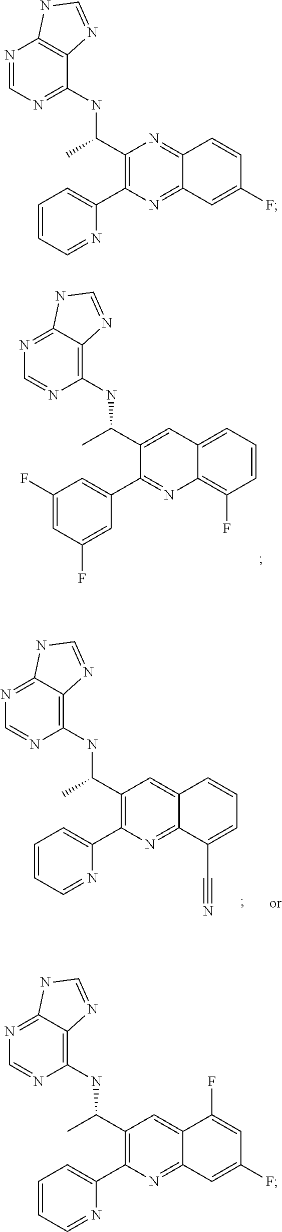

or a pharmaceutically acceptable salt thereof.

Also preferred is as a PI3K inhibitor is a compound of Formula IIa below, or a pharmaceutically acceptable salt thereof,

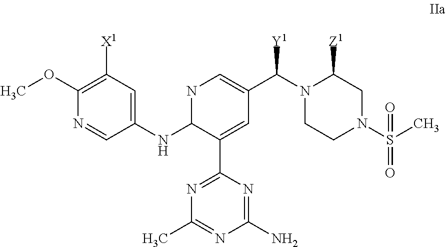

wherein X¹ is fluorine or hydrogen; Y¹ is hydrogen or methyl; and Z¹ is hydrogen or methyl. A particular PI3K inhibitor that can be used in the combinations is AMG 511 (also known as AMG 2539965 or 2539965), which is Example 148 of published PCT application WO2010/126895.

Other PI3K inhibitors that can be used in combination with Compound A in the combinations disclosed herein include Pan-PI3K inhibitors such as BKM120 and GDC-0941; PI3Kα selective inhibitors such as AMG 511 and BYL719; and PI3K β selective inhibitors such as GSK-2636771.

Compounds that inhibit both PI3K and mTOR (dual inhibitors) are known. In still another aspect, the present invention provides the use of dual PI3K and mTOR inhibitors for use in combination with KRAS^{G12C} inhibitors. An example of a particular dual inhibitor is GDC-0980.

mTOR is a protein in the PI3K pathway. It is another aspect of the present invention to use an mTOR inhibitor in combination with KRAS^{G12C} inhibitors. mTOR inhibitors that can be used in combination with Compound A include those disclosed in the following documents: PCT published application no. WO2010/132598 and PCT published application no. WO2010/096314. mTOR inhibitors that can be used in combination with Compound A include AZD2014 and MLN0128.

PKB (AKT) is also a protein in the PI3K pathway. It is another aspect to use an AKT inhibitor in combination with Compound A. AKT inhibitors that can be used include those disclosed in the following documents: U.S. Pat. Nos. 7,354,944; 7,700,636; 7,919,514; 7,514,566; U.S. patent application publication no. US 2009/0270445 A1; U.S. Pat. Nos. 7,919,504; 7,897,619; or PCT published application no. WO 2010/083246 A1. Particular AKT inhibitors that can be used in the combinations include MK-2206, GDC-0068 and AZD5363.

Compound A can also be used in combination with CDK4 and/or 6 inhibitors. CDK 4 and/or 6 inhibitors that can be used in the present combinations include, but are not limited to, those disclosed in the following documents: PCT published application no. WO 2009/085185 or U.S. patent application publication no. US2011/0097305.

Anti-PD-1 antibodies include, but are not limited to, Pembrolizumab (Keytruda™) Nivolumab, AUNP-12, AMG401, and Pidilizumab. Exemplary anti-PD-1 antibodies and methods for their use are described by Goldberg et al., Blood 110(1):186-192 (2007), Thompson et al., Clin. Cancer Res. 13(6):1757-1761 (2007), and Korman et al., International Application No. PCT/JP2006/309606 (publication no. WO 2006/121168 A1), each of which are expressly incorporated by reference herein.

KRAS^{G12C} inhibitors can also be used in combination with SHP2 inhibitors in the present invention. SHP2 inhibitors that can be used in the present combinations include, but are not limited to, RMC-4630 or RMC-4550 from Revolutions Medicines in Redwood City, CA.

The SHP-2 inhibitor may be an inhibitor disclosed in WO 2018/013597A1 (Revolution Medicines, Inc., published Jan. 18, 2018), US 2016/62361249, US 2017/62449523, WO 2016/203406 (Novartis, AG, published Dec. 22, 2016), and US 2015/62181893, which are each expressly incorporated by reference in their entireties for all purposes as if specifically set forth herein. In some cases, the SHP-2 inhibitor is SHP099. In some cases, the SHP-2 inhibitor is TNO155.

Compound A can be used in combination with the agents disclosed herein or other suitable agents, depending on the condition being treated. Hence, in some embodiments Compound A will be co-administered with other agents as described above. When used in combination therapy, Compound A is administered with the second agent simultaneously or separately. This administration in combination can include simultaneous administration of the two agents in the same dosage form, simultaneous administration in separate dosage forms, and separate administration. That is, Compound A and any of the agents described above can be formulated together in the same dosage form and administered simultaneously. Alternatively, Compound A and any of the agents described above can be simultaneously administered, wherein both the agents are present in separate formulations. In another alternative, Compound A can be administered just followed by and any of the agents described above, or vice versa. In some embodiments of the separate administration protocol, Compound A and any of the agents described above are administered a few minutes apart, or a few hours apart, or a few days apart.

### EXAMPLES

Compound A Dosing Study: patients identified with a cancer having a KRAS G12C mutation were enrolled in the study. The patients are adult patients with locally advanced or metastatic KRAS G12 C mutant solid tumors. All patients previously received prior standard therapies depending upon the tumor type and stage of disease. No patient exhibited active brain metastases. Patients in the dosing study had the following diagnoses: 14 with non-small cell lung cancer (NSCLC), 10 with colorectal cancer (CRC) and two with another KRAS G12 C mutant solid tumor. Compound A was administered orally once daily at the designated dose. Patients were given a dose of 180 mg, 360 mg, 720 mg, or 960 mg Compound A and adiographic scans were performed every six weeks.

Adverse events reported while taking Compound A are shown in Tables 1 and 2 below. Of the six serious adverse events reported, none was reported as related to Compound A. The six serious adverse events were two Grade 3 (1 Pneumonia, 1 Malignant biliary obstruction); one Grade 4 (Pericardial effusion); and three Fatal (1 Dyspnea; 2 Colorectal cancer metastatic). No patient reported any DLTs, Grade 4 related adverse events, or serious related adverse events.

TABLE 1
Treatment Emergent Adverse Event

| | | | | All |
|---|---|---|---|---|
| | Gr 1 | Gr 2 | Gr 3 | Grades |
| Adverse Event | n | n | n | n |
| Any Treatment Emergent | | | | 25 |
| Adverse Event | | | | |
| Decreased Appetite | 3 | 3 | 0 | 6 |
| Diarrhoea | 5 | 0 | 1 | 6 |
| Fatigue | 1 | 3 | 1 | 5 |
| Headache | 3 | 1 | 1 | 5 |
| Cough | 2 | 2 | 0 | 4 |
| Hot Flush | 4 | 0 | 0 | 4 |
| Nausea | 4 | 0 | 0 | 4 |

TABLE 2
Treatment Related Adverse Event

| | Gr 1 | Gr 2 | | Gr 1 | Gr 2 |
|---|---|---|---|---|---|
| Adverse Event | n | n | Adverse Event | n | n |
| Diarrhoea | 3 | | Flatulence | 1 | |
| Decreased appetite | 2 | | Vomiting | 1 | |
| Nausea | 2 | | Fatigue | 1 | |
| Elevated creatine | 1 | | Hyperkalemia | | 1 |
| phosphokinase | | | | | |
| Elevated or change | 1 | 1 | WBC Decrease | 1 | |
| in AST | | | | | |
| Elevated or change | 1 | 1 | Proteinuria | | 1 |
| in ALT | | | | | |
| Alkaline phosphatase | 1 | 1 | Pyrexia | 1 | |
| Cheilitis | | 1 | Arthralgia | 1 | |
| Dry mouth | 1 | | Hot Flush | 1 | |

| Grade 3 Adverse Event | n |
|---|---|
| Anemia | 1 |
| Diarrhea | 1 |

^{a} Patient had grade 2 anemia at baseline;
^{b} Lasting 2 days

Individual response to Compound A therapy: Case #1: A 61 year old woman diagnosed with KRAS G12C metastatic NSCLC in 2010, had prior therapy of Carboplatin/Taxol from August 2010 until October 2010; then Carboplatin/Pemetrexed from October 2016 until June 2017; then Nivolumab from August 2017 until April 2018; then was administered Compound A at a 180 mg dose. She exhibited a partial response at the 180 mg dose (−34%) at one of her six-week assessments. She has tolerated the drug and continues on it for more than 27 weeks.

Case #2: A 59 year old man diagnosed with a KRAS G12 C metastatic NSCLC in 2013, had prior therapy of Carboplatin/Pemetrexed February 2014 until February 2015; Erlotinib from April 2015 until June 2015; Nivolumab August 2015 until August 2017; Dasatinib from July 2016 until August 2017; M3541 (Targeted biologics) from October 2017 until November 2017; then was administered Compound A at a 360 mg dose. He exhibited a partial response (−80%) at one of his six-week assessments. He has tolerated the drug and continues on it for more than 14 weeks.

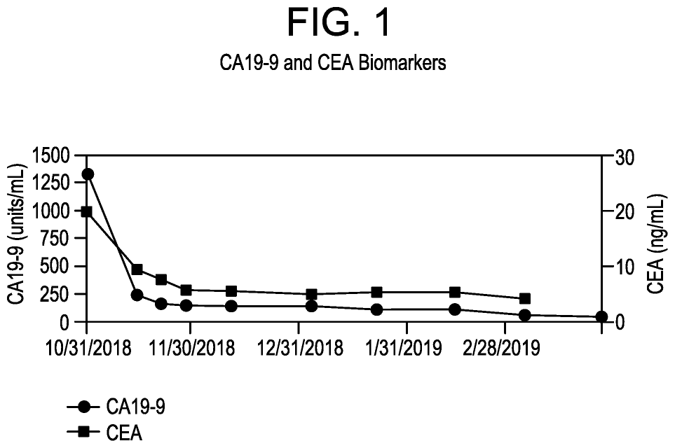

Case #3: A 34 year old woman diagnosed with KRAS G12C metastatic colon adenocarcinoma in 2014, had prior therapy of FOLFOX and HIPEC in August 2015, followed by FOLOX till December 2015; FOLFIRI with PD in August 2016; HIPEC October 2016; Capecitabine+bevacizumab August 2017; Phase I clinical trial March-June 2018; then was enrolled in October 2018 Phase I clinical trial for Compound A and administered Compound A at a dose of 360 mg. She exhibited a stable disease (−18%) at one of her six-week assessments. She also exhibited a biochemical response where her biomarkers CA 19-9 and CAE decreased rapidly upon administration of Compound A, and remained at the lower levels during the course of the Compound A therapy (FIG. 1). She has tolerated the drug and continues on treatment for more than 22 weeks.

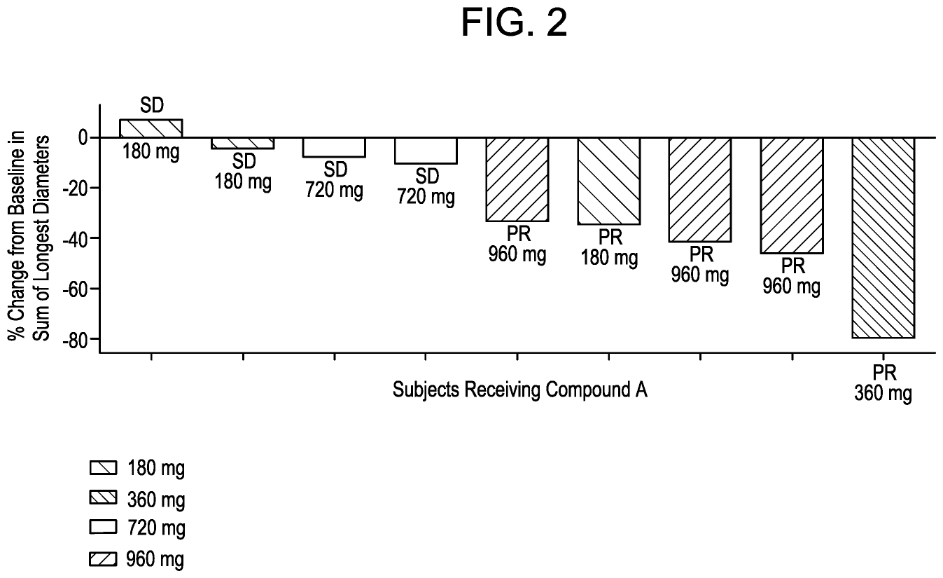

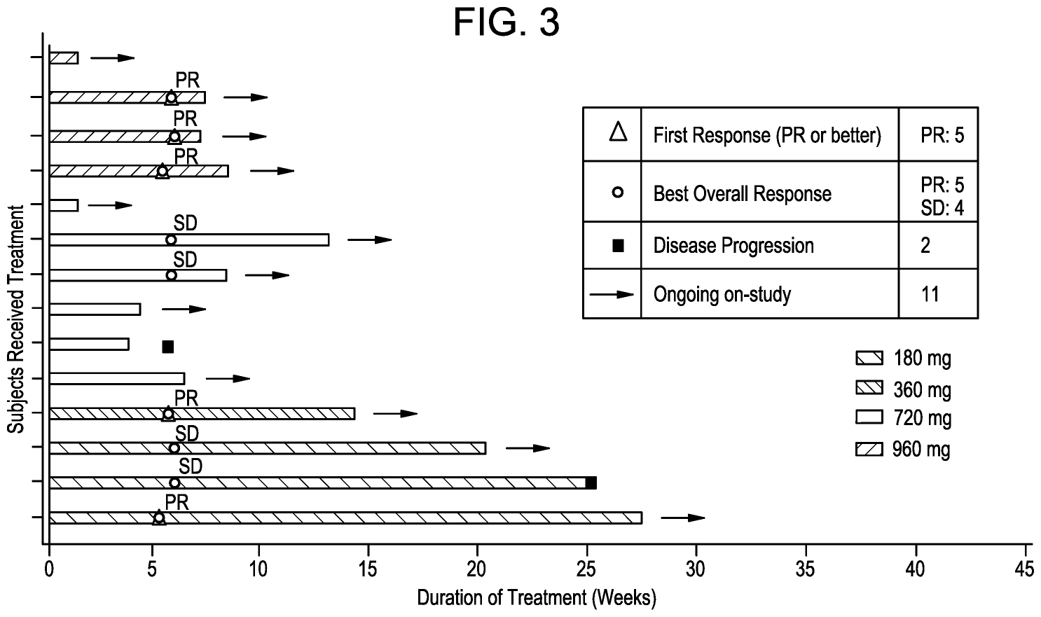

NSCLC Tumor Responses: Patients having KRAS G12C NSCLC were given a daily dose of 180 mg, 360 mg, 720 mg, or 960 mg, and 9 of the 10 patients studied exhibited at least a stable disease response to the therapy based upon radiographic scans performed every six weeks — results shown in FIG. 2 with designated dose noted below each histogram. Duration and treatment of the subjects in the NSCLC study are also shown in FIG. 3, where top four bars are for patients receiving a 960 mg total daily dose, next six bars are for patients receiving 720 mg total daily dose; next bar for patient receiving a 360 mg total daily dose, and bottom three bars for patients receiving a 180 mg total daily dose.

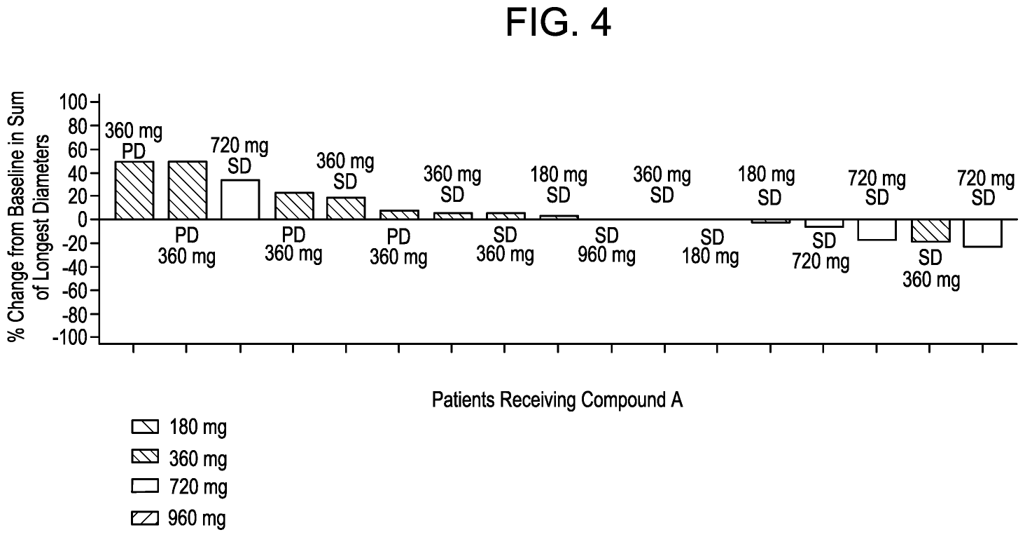

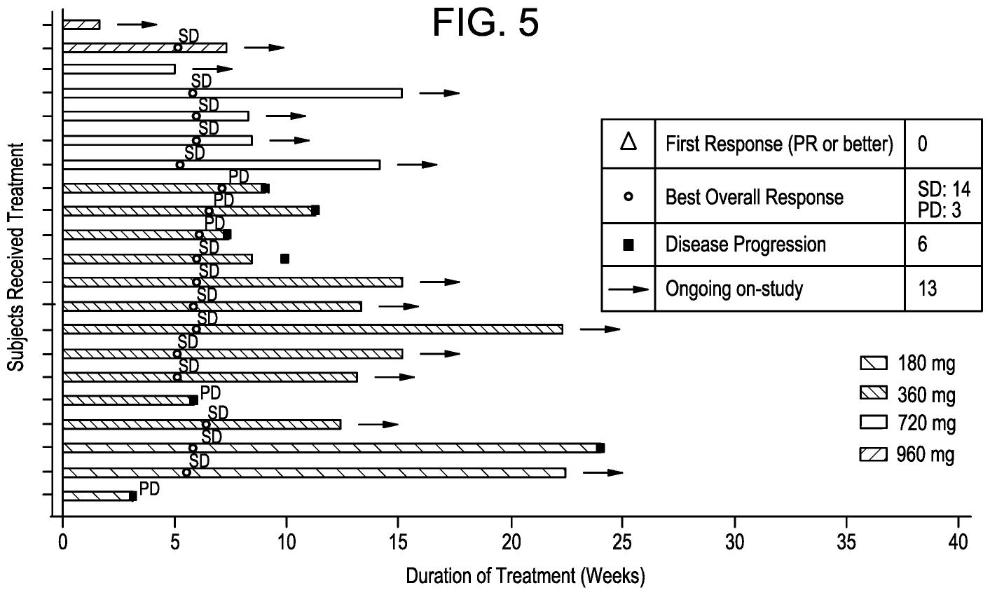

CRC and Other Solid Tumor Responses: Patients having a KRAS G12C CRC or other solid tumor were given a daily dose of 180 mg, 360 mg, 720 mg, or 960 mg, and results for 17 of the 19 patients studied are shown in FIG. 4 (the two not shown progressed prior to week 6 and were not subject to the first six-week assessment). The results in FIG. 4 are based upon radiographic scans performed at each six-week assessment. Duration and treatment of the subjects in the CRC/Other study are also shown in FIG. 5, where top two bars are for patients receiving a 960 mg total daily dose, next five bars are for patients receiving 720 mg total daily dose; next eleven bars for patients receiving a 360 mg total daily dose, and bottom three bars for patients receiving a 180 mg total daily dose.

Compound A Phase 1 Study Results: thirty-five patients having a KRAS G12C cancer (19 CRC, 14 NSCLC, 2 other—appendix) were enrolled in the phase 1 study for Compound A. All had 2 or more prior lines of therapy. No DLTs were reported. Sixteen patients reported Compound A—related adverse events, two with Grade 3 related adverse events (anemia and diarrhea). The best tumor responses were tabulated, and 26 patients remain on the study. Results are shown below in the Table.

Best Tumor Response in 29* Patients

| | | Duration of Response or |
|---|---|---|
| | Frequency | Stable Disease** |
| NSCLC | | |
| (n = 10) | | |
| Partial Response | 5 | 7.3-27.4 weeks |
| | (2 confirmed) | |
| Stable Disease | 4 | 8.4-25.1 weeks |
| Progressive | 1*** | n/a |
| Disease | | |
| CRC/Other | | |
| (n = 19) | | |
| Stable Disease | 14 | 7.3-24.0 weeks |
| Progressive | 5*** | n/a |
| Disease | | |

*Six pts (4 NSCLC; 2 CRC/Other) did not have a post-baseline radiographic scan as of the data cutoff date (4 Apr. 2019)
**Duration of response as of the data cutoff dare. All 5 pts with partial response are still on treatment as of the data cutoff date.
***Two of these pts (1 NSCLC; I CRC) had early (prior to week 6) clinical progressive disease.

The pharmacokinetics of a Compound A 960 mg orally administered dose is as follows: Cₘₐₓ of 7.84 μg mL (SD of 8.09); AUC_{0-24 hr} 140 hr*μg/mL (SD of 117); and t_{1/2,z} 6.5 hr (SD of 4.2-8.0).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What is claimed:

1. A method of treating cancer, the method comprising: administering to a subject in need thereof Compound A in a daily dose of 180 mg, 360 mg, 720 mg, or 960 mg, wherein Compound A has the following structure 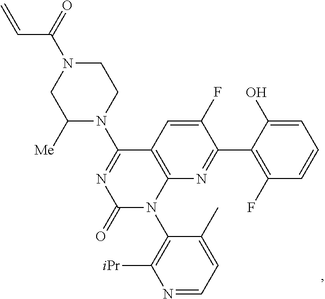 [SMILES: C=CC(=O)N1CCN(c2nc(=O)n(-c3c(C)ccnc3C(C)C)c3nc(-c4c(O)cccc4F)c(F)cc23)C(C)C1]
or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;
in combination with a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof.

2. The method of claim 1, wherein Compound A has the structure

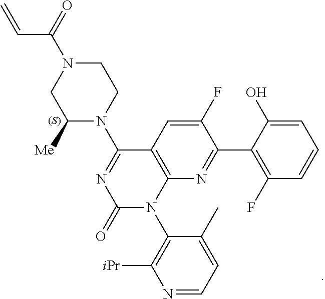

3. The method of claim 1, wherein Compound A has the structure

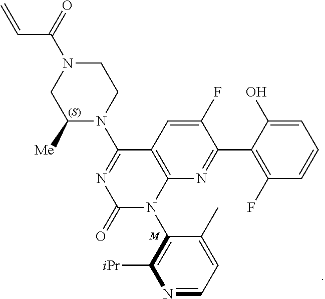

4. The method of any one of claim 1, 2 or 3, wherein the SHP2 inhibitor is RMC-4630.

5. The method of any one of claims 1-4, wherein the SHP2 inhibitor is orally administered.

6. The method of any one of claims 1-5, wherein the cancer is a solid tumor.

7. The method of any one of claims 1-6, wherein the cancer is non-small cell lung cancer.

8. The method of any one of claims 1-6, wherein the cancer is colorectal cancer.

9. The method of any one of claims 1-6, wherein the cancer is pancreatic cancer.

10. The method of any one of claims 1 to 9, wherein the cancer is a KRAS G12C mutated cancer.

11. The method of any one of claims 1 to 10, wherein the subject, prior to start of Compound A therapy, had undergone at least one other systemic cancer therapy.

12. The method of claim 10, wherein the subject had undergone at least two other systemic cancer therapies.

13. The method of any one of claims 1 to 10, wherein Compound A is administered orally.

14. The method of any one of claims 1 to 13, wherein the Compound A is administered as a single daily dose.

15. The method of any one of claims 1 to 13, wherein the Compound A is administered as a twice a day dose.

16. The method of any one of claims 1 to 15, wherein the Compound A dose is 180 mg.

17. The method of any one of claims 1 to 15, wherein the Compound A dose is 360 mg.

18. The method of any one of claims 1 to 15, wherein the Compound A dose is 720 mg.

19. The method of any one of claims 1 to 15, wherein the Compound A dose is 960 mg.

20. The method of any one of claims 1 to 19, wherein the subject is administered Compound A for at least one month.

21. The method of any one of claims 1 to 19, wherein the subject is administered Compound A for at least three months.

22. The method of any one of claims 1 to 19, wherein the subject is administered Compound A for at least six months.

23. The method of any one of claims 20 to 22, wherein the subject exhibits at least a stable disease (SD).

24. The method of any one of claims 20 to 22, wherein the subject exhibits at least a partial response (PR).

25. The method of any one of the claims 1-24, wherein the Compound A is administered before the SHP2 inhibitor.

26. The method of any one of the claims 1-24, wherein the Compound A is administered concurrently with the SHP2 inhibitor.

27. The method of any one of the claims 1-24, wherein the Compound A is administered after the SHP2 inhibitor.

28. A pharmaceutical composition comprising: a Compound A, wherein Compound A has the following structure 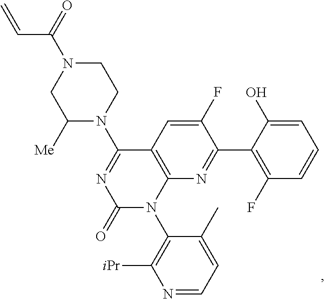 [SMILES: C=CC(=O)N1CCN(c2nc(=O)n(-c3c(C)ccnc3C(C)C)c3nc(-c4c(O)cccc4F)c(F)cc23)C(C)C1]
or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;
and a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof.

29. The composition of claim 28, wherein Compound A has the structure

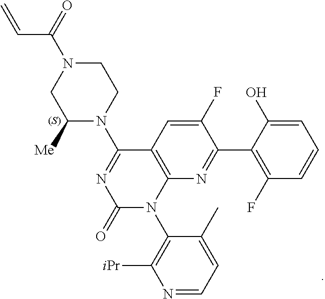

30. The composition of claim 28, wherein Compound A has the structure

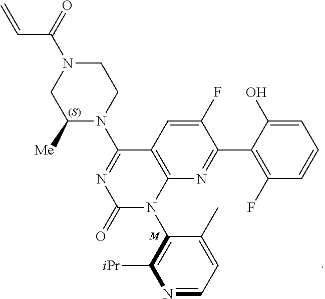

31. A kit for treating cancer, the kit comprising: a Compound A wherein Compound A has the following structure 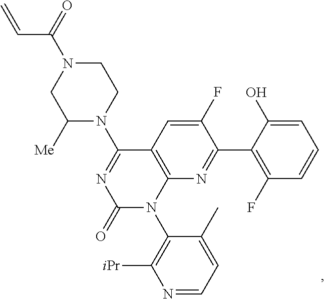 [SMILES: C=CC(=O)N1CCN(c2nc(=O)n(-c3c(C)ccnc3C(C)C)c3nc(-c4c(O)cccc4F)c(F)cc23)C(C)C1]
or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;
and a SHP2 inhibitor, or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;

32. The kit of claim 31, wherein Compound A has the structure

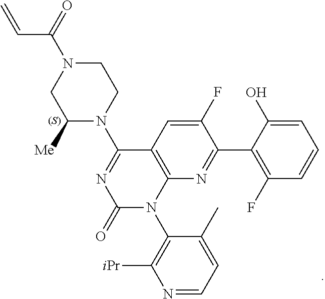

33. The kit of claim 31, wherein Compound A has the structure

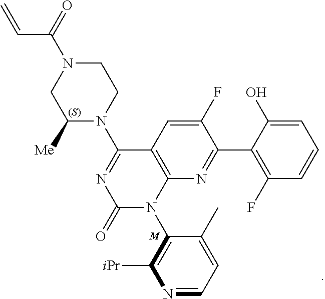

34. The kit of any one of claims 31-33, wherein the cancer is a solid tumor. cancer.

35. The kit of any one of claims 31-33, wherein the cancer is non-small cell lung

36. The kit of any one of claims 31-33, wherein the cancer is colorectal cancer.

37. The kit of any one of claims 31-33, wherein the cancer is pancreatic cancer.

38. The kit of any one of claims 31-33, wherein the cancer is a KRAS G12C mutated cancer.
